(19) United States
(12) Patent Application Publication
(10) Pub. No.: US 2021/0403870 A1
(43) Pub. Date: Dec. 30, 2021

(54) Methods and Systems for In Vitro Cardiac Disease Modeling

(71) Applicant: The Medical College of Wisconsin, Inc., Milwaukee, WI (US)

(72) Inventors: Ulrich Broeckel, Wauwatosa, WI (US); Praful Aggarwal, Wauwatosa, WI (US); Milica Radisci, Toronto (CA); Yimu Zhao, Toronto (CA)

(21) Appl. No.: 17/297,075

(22) PCT Filed: Nov. 25, 2019
(86) PCT No.: PCT/US19/62976

§ 371 Date: May 26, 2021

Related U.S. Application Data
(60) Provisional application No. 62/772,428, filed on Nov. 28, 2018.
(60) Provisional application No. 62/885,948, filed on Aug. 13, 2019.

(51) Int. Cl. C12N 5/077

(52) U.S. Cl. CPC C12N 5/0657; C12N 2529/00

(57) ABSTRACT

A method for generating an in vitro cardiac tissue model. The method includes steps of: forming an elongated tissue by disposing a plurality of cardiomyocytes within a culture plate; culturing the tissue such that each end of the elongated tissue contacts one of a pair of attachment wires adhered to the culture plate; and electrically stimulating the elongated tissue in culture.

### CROSS-REFERENCE TO RELATED APPLICATIONS

This application claims the benefit of priority from U.S. Provisional Patent Application Ser. No. 62/772,428, filed Nov. 28, 2018, and 62/885,948, filed Aug. 13, 2019, the entire disclosures of which are incorporated herein by reference.

### STATEMENT REGARDING FEDERALLY SPONSORED RESEARCH

121 This invention was made with government support under HL107437 awarded by NHLBI. The government has certain rights in the invention.

### FIELD OF THE INVENTION

This document relates to methods, systems, and apparatus for generating an in vitro model system for studying chronic heart disease.

### BACKGROUND

Induced pluripotent stem cells (iPSC) offer the possibility to determine the pathogenesis of cardiac disease, as has been powerfully demonstrated with cardiac microtissues used to model cardiomyopathy as a result of sarcomeric protein titin truncations or mitochondrial protein taffazin mutations. Nevertheless, some of the most common cardiac diseases are complex, polygenic conditions that are strongly influenced by environmental factors. For example, hypertensive heart disease reflects the cardiac changes induced by prolonged hypertension leading to cardiac hypertrophy, left ventricular dysfunction, and ultimately heart failure. However, current in vitro models fail to adequately reproduce the conditions that lead to chronic heart disease.

### SUMMARY OF THE PRESENT DISCLOSURE

Thus, to model polygenic disease, it may be helpful to provide a chronic increased workload to the cardiac tissue over a prolonged time period and/or to stress the tissue with a shorter but less preferred culture condition. Accordingly, the present disclosure provides apparatus, methods, and systems for providing a weeks- to months-long biophysical stimulation of 3D tissues to model a polygenic disease. In certain embodiments of the present disclosure, the platform disclosed herein enables formation or manufacture of thin, cylindrical tissues, similar to human trabeculae, suspended between two parallel polymer wires whose deflection can be used to quantify passive and active forces of the tissues. Tissue grown in vitro with this platform may be subjected to electrical stimulation over a period of time, such as weeks or months, which provides a chronic increased workload (e.g. by providing a physiological beat rate resembling human cardiac contraction frequencies and allowing for a controlled adjustment of workload and stress) that causes the tissue to mimic diseased cardiac tissue.

Thus, in one embodiment the invention provides a method for generating an in vitro cardiac tissue model. The method includes steps of: forming an elongated tissue by disposing a plurality of cardiomyocytes within a culture plate; culturing the tissue such that each end of the elongated tissue contacts one of a pair of attachment wires adhered to the culture plate; and electrically stimulating the elongated tissue in culture.

In another embodiment, the invention provides a kit for generating an in vitro cardiac tissue model. The kit includes a culture system including a culture plate and a pair of attachment wires; and a plurality of hiPSC-derived cardiomyocytes from at least one of a human subject with evidence of a cardiac disease and a human subject without evidence of a cardiac disease.

In yet another embodiment, a culture system for cardiac disease modeling, including: a culture plate including a pair of anchor mechanisms; and a plurality of hiPSC-derived cardiomyocytes from at least one of a human subject with evidence of a cardiac disease and a human subject without evidence of a cardiac disease.

In still another embodiment, a human induced pluripotent stem cell (hiPSC) from a human subject with evidence of a cardiac disease selected from the group consisting of: Affected D (no. A2637), Affected E (no. A2614), and Affected F (no. A2779).

In yet another embodiment, a human induced pluripotent stem cell (hiPSC) from a human subject without evidence of a cardiac disease selected from the group consisting of: Non-Affected A (no. A7156), Non-Affected B (no. 50000395), and Non-Affected C (no. U2474).

The foregoing and other aspects and advantages of the present disclosure will appear from the following description. In the description, reference is made to the accompanying drawings that form a part hereof, and in which there is shown by way of illustration one or more exemplary versions. These versions do not necessarily represent the full scope of the invention.

### BRIEF DESCRIPTION OF THE DRAWINGS

The following drawings are provided to help illustrate various features of example embodiments of the disclosure, and are not intended to limit the scope of the disclosure or exclude alternative implementations.

FIG. 1A shows steps for generating in vitro cardiac tissue using the Biowire II platform, which generates micro-scale engineered cardiac tissues in a low-absorption environment. A) Schematic of the Biowire II platform design and tissue generation;

FIG. 1B shows a series of photos of an example of cardiac tissue formed using the Biowire II platform, showing a culture dish (left panel), an inset showing the microwells (middle panel), and a further inset showing the tissue attached to the attachment wires (POMaC wires);

FIGS. 2A-2H demonstrate that the Biowire II platform enables proof of concept cardiac disease modelling based on a comparison between two disease cell lines Control C (less severe; also referred to herein as “Non-Affected C”) and Affected F (more severe); FIG. 2A shows that baseline expression analysis in Affected F showed a significant enrichment in cardiac dysfunction when compared to Control C as determined by IPA Tox List analysis; FIG. 2B shows that tissue compaction rates were at the same level in the first week; FIGS. 2C-2G show that tissues created from Control C tended to have: higher excitation threshold (ET) (FIG. 2C), lower maximum capture rate (MCR) (FIG. 2D), higher force of contraction (FIG. 2E), faster contraction (FIG. 2F), and faster relaxation (FIG. 2G) than tissues created from Affected F (data presented as mean±stdev, n≥7, two-way ANOVA with Tukey post hoc multiple comparison test); FIG. 2H shows confocal images of Control C and Affected F tissues immunostained for sarcomeric α-actinin, F-actin stain, and counterstained with the nuclear stain DAPI, scale bar=50 μm;

FIG. 3A shows a summary of clinical features of the subjects who were sources of the cardiomyocytes (CMs) used for the experiments disclosed herein, including patients with clear echocardiographic evidence of left ventricular hypertrophy (Affected) versus participants without ventricular hypertrophy (Non-Affected, sometimes referred to as “Control”) indicating PublicationID, Gender, Age, Hypertrophy Index (lvmht27), and ejection fraction (EF) of hypertensive patients contributing iPSCs;

FIG. 3B shows a graph of a long-term electrical conditioning protocol used to mimic chronic increased workload in ventricular tissues created from patient iPSC-CMs: tissues were first subjected to a ventricular 1 Hz step-up electrical conditioning protocol and once the stimulation frequency of 6 Hz was reached and applied for a week, it was decreased to 3 Hz and maintained at that level for up to 6 months. Biowires (i.e. in vitro cardiac tissues) generated with iPSCs derived from hypertensive patients with evidence of heart disease (Affected D, E and F) were compared to Biowires of patients with no evidence of heart disease (Non-Affected A, B and C).

FIG. 3C shows the results from two independent experiments, using Biowires from Non-affected A, B vs. Affected D, E and Non-affected C vs. Affected F, which were analyzed by Gene Set Enrichment Analysis (GSEAs); these experiments reveal enrichment in Affected patients for cardiac genes associated with cardio-functional categories and cardiac related canonical pathways, determined by IPA Tox List analysis;

FIG. 3D shows a Venn diagram indicating the overlap of enriched signaling pathways related to cardiotoxicity from both experiments. The functional categories shown have a Benjamini-Hochberg multiple correction p-value≤0.05;

FIG. 3E shows a heat map showing a sub-set of genes related to cardiac hypertrophy;

FIG. 3F shows that active force was significantly reduced in the tissues derived from the patients that exhibited a higher level of left ventricular hypertrophy in response to a prolonged hypertension (Affected D vs. Affected E, p=0.0387) compared to the Non-Affected patient (non-affected A vs. Affected D, p=0.0006; non-affected A vs. Affected F, p=0.0023; Non-affected B vs. Affected D, p=0.0382) at the 6 week culture period (one way ANOVA with Tukey's multiple comparisons test);

FIG. 3G shows that active force was absent in all tissues from Affected patients (Affected D, E and F) compared to the Non-Affected patients (non-affected A, B, and C) after an 8 month culture period;

FIG. 3H shows live (green) and dead (red) staining of tissues at the end of an 8 month culture period. Viability was quantified with no significant differences among the groups. Scale bar=100 μm;

FIG. 3I shows confocal images and quantification of the presence of sarcomeric α-actinin (green) counterstained with DAPI (Blue), Scale bar=30 μm (one way ANOVA with Tukey's multiple comparisons test);

FIG. 4A shows hierarchical clustering of the gene expression results from Biowires generated from iPSC-CMs provided by 3 Non-Affected and 3 Affected patients;

FIG. 4B shows additional results from GSEAs for Biowires which reveal enrichment in Affected patients for cardiac genes associated with heart disease and arrhythmias, determined by IPA Tox List analysis;

FIG. 4C top panels show live (green) and dead (red) staining of tissues at the end of 8 month culture period for Control C (left) and Affected F (right) cells. Scale bar=100 μm; lower panels show confocal images of sarcomeric α-actinin (green) counterstained with DAPI (Blue), Scale bar=30 μm;

FIG. 4D shows that active force was absent in the tissues derived from a patient that exhibited a higher level of left ventricular hypertrophy in response to a prolonged hypertension (Affected F) compared to the less affected patient (Control C, p=0.1147);

FIG. 4E shows that active force was significantly reduced or absent in the tissues derived from the other two patients that exhibited a higher level of left ventricular hypertrophy in response to a prolonged hypertension (Affected D and Affected E, p=0.0241, Student's t-test) compared to the less affected patient (Control A, p=0.0208, and Control B, p=0.2035, Student's t-test) at the end of 5 month culture period.

### DETAILED DESCRIPTION OF THE PRESENT DISCLOSURE

In various embodiments, a method and system are disclosed which provide a model system for studying cardiac diseases, particularly polygenic diseases such as left ventricular hypertrophy, starting from various cell sources including patient cells. The model system is based on the formation of engineered cardiac tissues (ECTs) from stem cells, in particular hiPSC-derived cardiomyocytes, which are then cultured over a period of time (e.g. weeks to months) while being stimulated to contract so as to induce a disease state in the tissue. In preferred embodiments, the model system is based on a Biowire platform and the cardiac tissue that is formed using this platform is referred to as a Biowire.

Generating Heart Tissues from Multiple Cell Sources

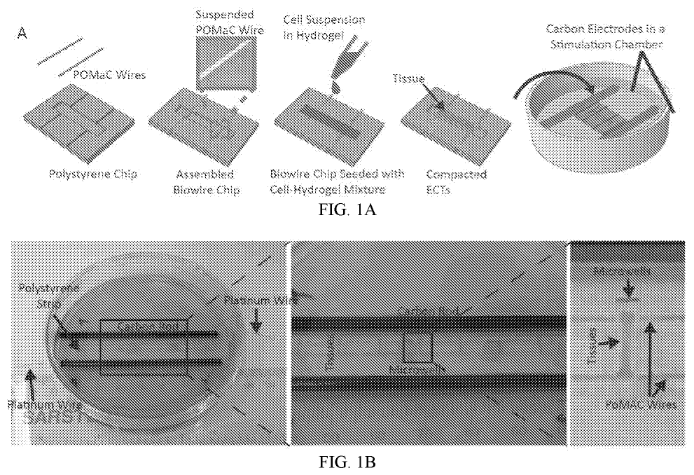

FIGS. 1A and 1B illustrate the general features of a Biowire platform (specifically the Biowire II platform), which in various embodiments may include one or more microwells (e.g. 5 mm×1 mm×0.3 mm) patterned onto a polystyrene chip or sheet that serves as a culture plate. Two flexible wires, e.g. manufactured from a POMaC polymer, may be secured (e.g. with adhesive glue) along either end of each elongated microwell. In certain embodiments, myocardial tissues may be created by combining ˜100,000 cardiomyocytes (CMs) and cardiac fibroblasts (e.g. at a 10:1 ratio) with hydrogel within each microwell. During the next ˜7 days, the cells generally undergo “compaction” thereby forming cylindrical trabecular strips (referred to as Biowires) that are suspended in the microwell but physically attached to the POMaC wires (see FIGS. 1A and 1B). Beginning at 1 week following initial assembly, the suspended Biowires may be electrically conditioned for a period of time (e.g. weeks) with electrical field stimulation via a pair of carbon electrodes connected to a stimulator with platinum wires (FIG. 1B). Details of embodiments of the conditioning protocols used are described below. A typical Biowire created using ventricular CMs from stem cells (e.g. BJ1D stem cells) may display uniform longitudinal alignment of sarcomeric contractile proteins after 6 weeks in culture. Additional information regarding the Biowire platform are disclosed in US Pat. Appl. Publ. No. 2016/0282338 entitled “Compositions and Methods for Making and Using Three-Dimensional Issue Systems,” which is incorporated by reference herein in its entirety for all purposes.

In some embodiments, one or more other apparatuses may be used to grow and/or test engineered heart tissue (see: Mannhardt et al., Stem Cell Reports 7:29-42 (2016); Lemoine et al., Scientific Reports 7:5464 (2017); Leonard et al., J. Molecular Cellular Cardiology 118:147-158 (2018); Feinberg et al., Stem Cell Reports, 1:387-396 (2013); each of which is incorporated by reference in its entirety). In various embodiments, a plurality of cardiomyocytes (e.g. derived from hiPSCs) may be cultured in a culture plate under suitable conditions to form an elongated segment of engineered heart tissue. One or both ends of the engineered heart tissue may be attached to an anchor or support mechanism such as a rod, wire, post, or other suitable attachment. The engineered heart tissue may then be stimulated over time, for example by one or more electrodes placed adjacent to the tissue, and force generated by the tissue may be monitored, e.g. using force transducer(s) associated with one or both anchor/support mechanisms and/or by tracking movement of one or both anchor/support mechanisms.

Procedures for producing induced pluripotent stem cells are described in Yu et al. (2007) [ref. 6] and Yu et al. (2009) [ref. 7], each of which is incorporated by reference herein in its entirety. Procedures for producing cardiomyocytes from stem cells are described in Kehat et al. [ref. 8], Xu et al. [ref. 9], He et al. [ref. 10], Mummery et al. [ref. 11], Sartiani et al. [ref. 12], Satin et al. [ref. 13], Zhang et al. [ref. 14], and Zwi et al. [ref. 15], each of which is incorporated by reference herein in its entirety.

Long-Term Culture with Chronic Increased Workload

In certain embodiments a disease state was induced in the Biowire cardiac tissue by repeatedly stimulating the tissue to contract over an extended period of time, for example over weeks or months, as described below. As an initial step, the Biowire ECTs may be prepared using patient-derived cell lines, as described below.

In certain embodiments Biowire ECTs were generated from Control A (also referred to as Non-Affected A—no. A7156), Control B (also referred to as Non-Affected B—no. 50000395), Control C (also referred to as Non-Affected C—no. U2474), Affected D (no. A2637), Affected E (no. A2614), and Affected F (no. A2779) cardiomyocytes, provided by Cellular Dynamics Inc., Madison, Wis. Code numbers cited herein identify hiPSC cell lines that were used to generate the cardiomyocytes. The codes correspond to identifiers contained in the Database of Genotypes and Phenotypes (dbGaP), which is a National Institutes of Health (NIH) sponsored repository that archives, curates, and distributes information produced by studies investigating the interaction of genotype and phenotype. Collagen hydrogel was used to generate ECTs. Each ECT contained 0.1 million CM. In some embodiments, an additional 5% of mesenchymal stem cells (MSC) were added as the side population to enhance tissue compaction and cell alignment. In certain embodiments, electrical stimulation started at 2 Hz on day 7 and the protocol of 1 Hz daily step-up was used until the frequency reached 6 Hz. ECTs were cultured in plating medium (Cellular Dynamics) for the initial week and then switched to maintenance media (Cellular Dynamics).

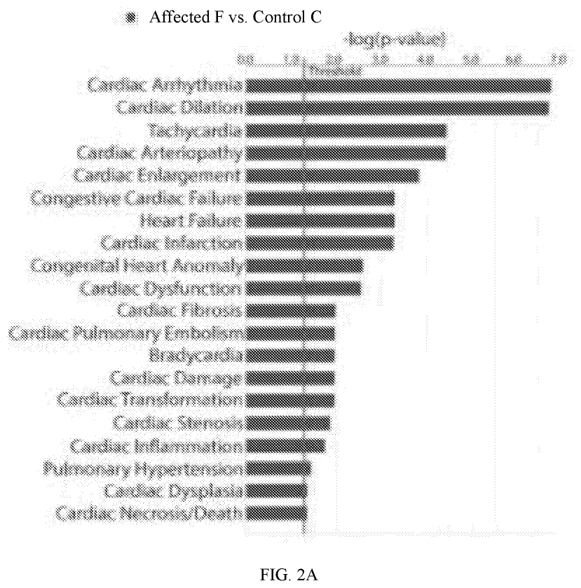

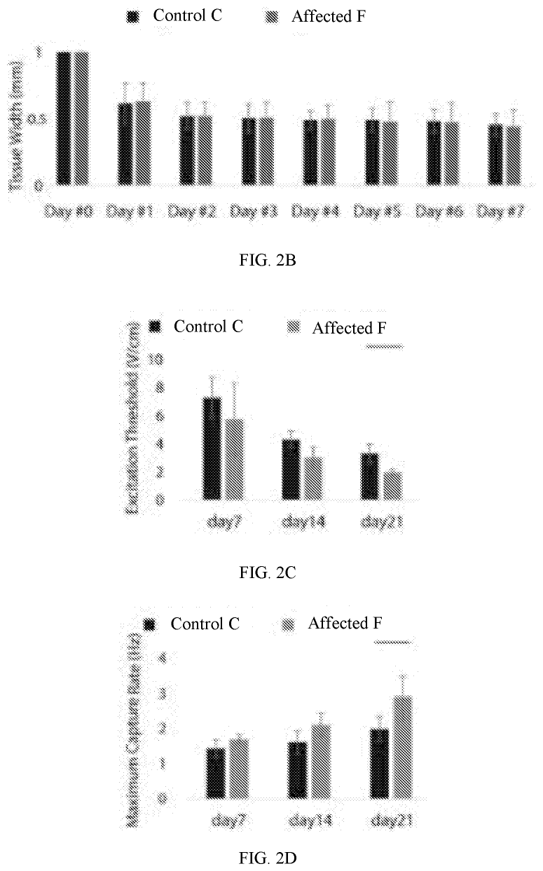

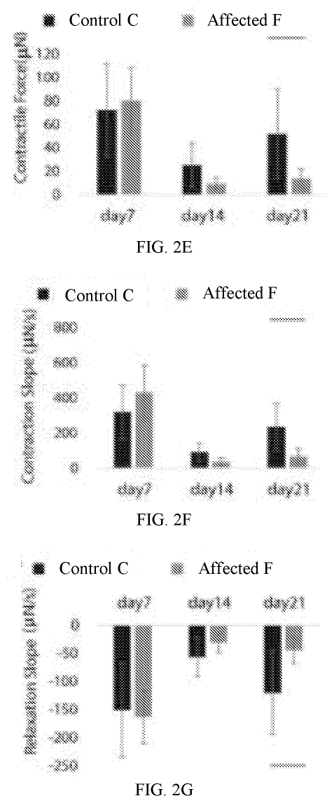

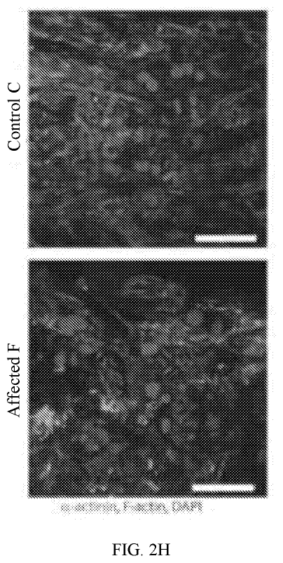

Strips of polystyrene containing eight microwells were transferred to a 10 cm tissue culture dish (FIGS. 1A and 1B). The strip surface was rinsed with 5% (w/v) Pluronic Acid (Sigma-Aldrich) and then air dried in a biosafety cabinet. In some embodiments, cardiac myocyte cells and cardiac fibroblasts (LONZA, Clonetics™ NHCF-V) were mixed in a 10:1 cell number ratio, pelleted, and resuspended at a concentration of 5.5×10⁷ cells/mL (unless otherwise specified) in a hydrogel. For long-term ventricular disease model preparation, tissues were generated from Control A, Control B, Control C, Affected D, Affected E, and Affected F cardiomyocytes. In various embodiments, electrical stimulation started at 2 Hz on day 7 post cell seeding and a protocol of 1 Hz weekly step-up was used until the frequency reached 6 Hz, at which point it was maintained at 6 Hz for one week. Subsequently, the frequency was decreased to 3 Hz and maintained at that level for the remainder of the cultivation period, which in certain embodiments was up to 6 months (see FIG. 2B). In certain embodiments, tissue were assessed after 6 Hz stimulation was reached (6 weeks) and after 5 months (Control A, Control B, Affected D and Affected E) or after 8 months (Control C and Affected F) of total culture period.

Gene Expression for Patient Derived Cells

Gene expression of ventricular tissues based on two individual human iPSC cardiomyocyte (hiPSC-CM) cell lines (Affected F and Control C) at the end of cultivation (e.g. after 8 months) with electrical conditioning, was assessed as previously described (3). Whole transcriptome sequencing was performed utilizing the Ion Total RNA-Seq Kit and the Ion Torrent Proton System (Thermofisher Scientific) following the manufacturer's recommendations. Data analysis was performed using Qiagen's Ingenuity Pathway Analysis (IPA) software with the overlay tool IPATox. The feature “Tox List” was set at default parameters to analyze genes contributing to principle component analysis between affected F and control C, focusing on cardiotoxicity.

Active and Passive Force for Cardiac Tissues

In certain embodiments, forces exerted by the Biowire ECTs were assessed by monitoring movements of the POMaC wires by measuring POMaC wires' autofluorescence. The POMaC wires were illuminated with ultraviolet light (e.g. λₑₓ=350 nm) and the autofluorescence was monitored in the blue region (blue channel) of the visible spectrum (e.g. λₑₘ 470 nm).

Blue channel image sequences were analyzed using a custom MatLab code that traced the maximum deflection of the POMaC wire. Average tissue width (diameter) and width of the tissue on the polymer wire (Tw) were measured from still frames of the 4× bright field video of the tissue in the relaxed position. Total (at peak contraction) and passive (at rest) POMaC wire deflections were converted to force measurements (μN) using the force calibration curves described in the previous section. The final readouts for the total and passive tension of tissue were then interpolated according to the Tw and custom tip sizes. The active force was calculated as the difference between the total and passive tension. The custom MatLab code was used to calculate the passive tension, active force, contraction and relaxation duration, and upstroke, and relaxation velocity.

Immunostaining and Confocal Microscopy

For live and dead staining, fresh working solutions were diluted from stock CFDA (10 mM, Invitrogen C1157) (1:1000 dilution) and PI (1 mg/mL P1304MP) (75:1000 dilution). Tissues from culture were washed with PBS and incubated in the working solution at 37° C. for 30 minutes. After being washed twice with fresh PBS, tissues were imaged with an Olympus FluoView 1000 laser scanning confocal microscope.

For immunostaining, tissues were fixed with 4% paraformaldehyde overnight first, permeabilized with 0.2% Tween20, and then blocked with 10% FBS. Immunostaining was performed using the following primary antibodies: mouse anti-cardiac Troponin T (cTnT) (ThermoFisher; 1:200), rabbit anti-Connexin 43 (Cx-43) (Abcam; 1:200), mouse anti-α-actinin (Abcam; 1:200), rabbit anti-myosin light chain-2v (Santa Cruz; 1:200), goat anti-caveolin3 (Santa Cruz; 1:100); and the following secondary antibodies: donkey anti-mouse-Alexa Fluor 488 (Abcam; 1:400), donkey anti-rabbit-Alexa Fluor 594 (Life Technologies; 1:200), and donkey anti-goat-Alexa Fluor 647 (Life Technologies; 1:200). Phalloidin-Alexa Fluor 660 (Invitrogen; 1:200) was used to stain F-actin fibers. Conjugated vimentin-Cy3 (Sigma; 1:200) was used to stain for vimentin. Confocal microscopy images were obtained using an Olympus FluoView 1000 laser scanning confocal microscope (Olympus Corporation).

In Vitro Disease Modeling

Short-Term Culture

In order to better describe the starting cell population, the hiPSC-CMs lines were characterized prior to seeding into the Biowire II platform. Expression profiling was conducted using whole transcriptome sequencing data. Baseline expression levels showed a significant enrichment in cardiac dysfunction in cell line Affected F when compared to Control C (FIG. 2A).

Instead of the long-term culture conditions described below, for initial characterization mesenchymal stem cells were used as a side population instead of cardiac fibroblasts, and conventional culture media provided with the cells from the supplier (Cellular Dynamics) was used instead of the optimized media described below. It was expected that the cardiomyocytes grown under such culture conditions would experience significant stress compared to the protocol described below. The ventricular tissues generated from these lines compacted at a similar rate (FIG. 2B). The Affected F tissues had lower excitation thresholds (ET) and higher maximum capture rates (MCR) during maturation (FIGS. 2C, 2D). The contractile force on day 7, prior to electrical stimulation, was similar for both cell lines but began to diverge after only 1 week of electrical stimulation, such that the Affected F tissues generated significantly less force than the Control C tissues on day 21 (FIG. 2E). The contraction and relaxation velocity were also significantly reduced in the Affected F tissues on day 21 (FIGS. 2F, 2G) and the structure of the Affected F tissues was irregular and disordered (FIG. 2H), relative to the Control C tissues.

Long-Term Culture

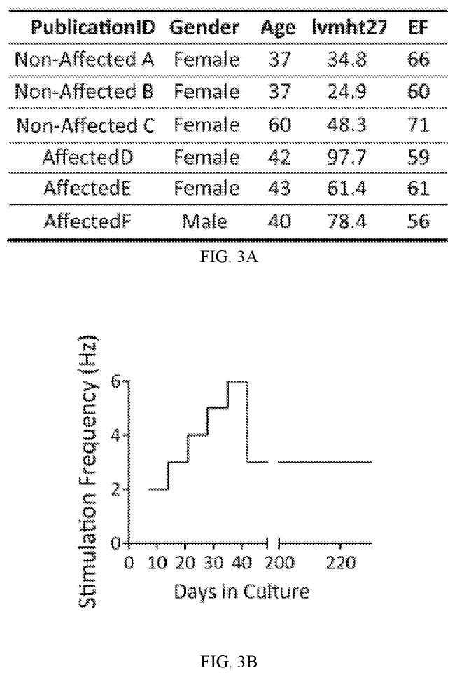

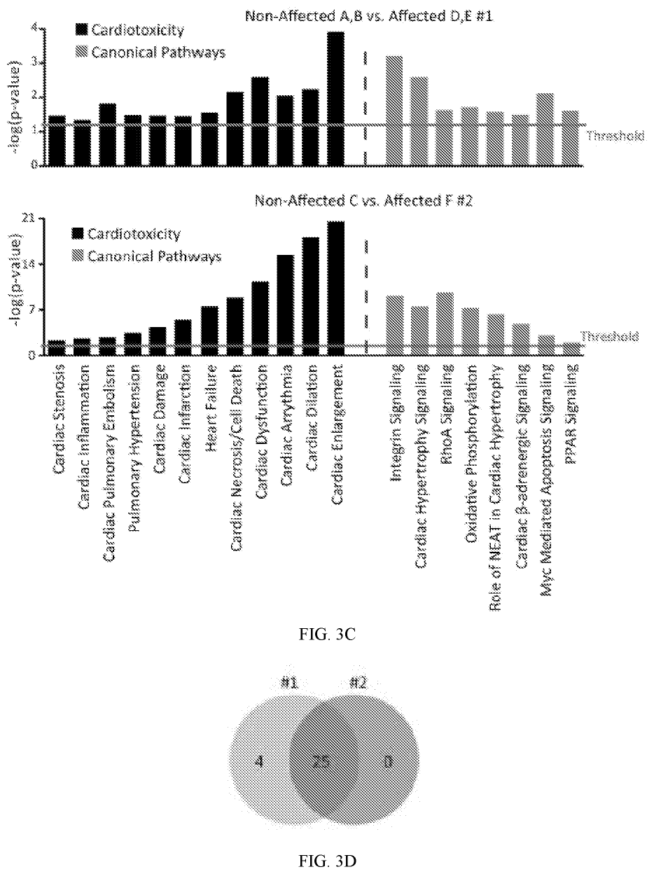

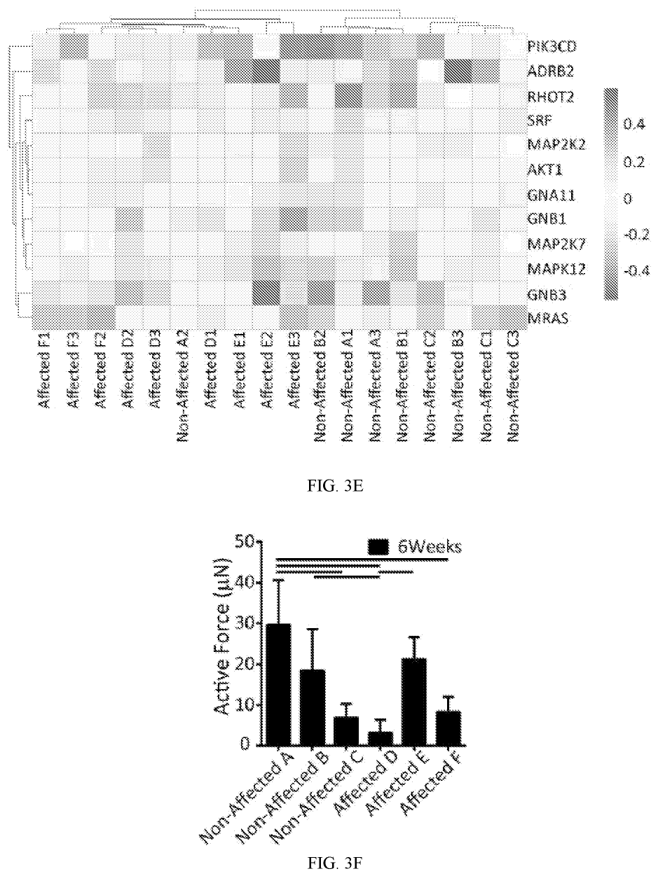

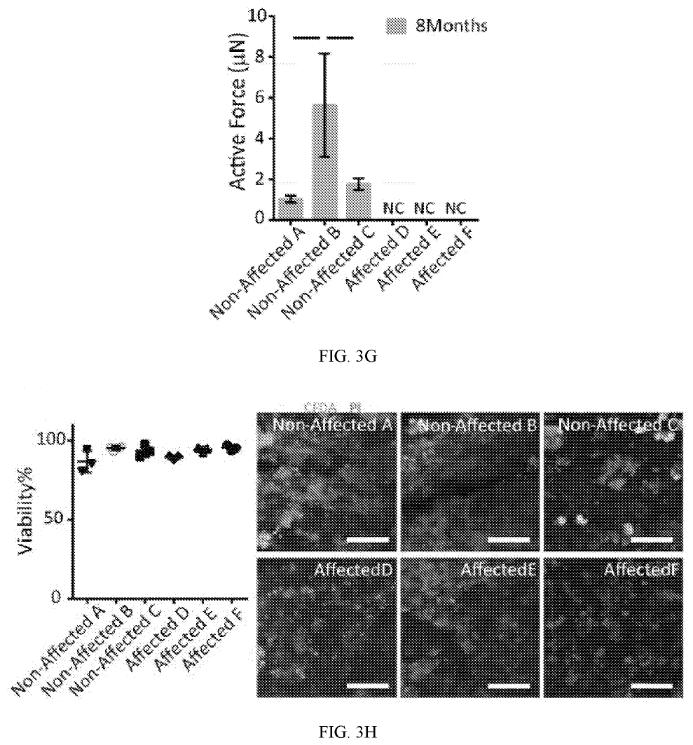

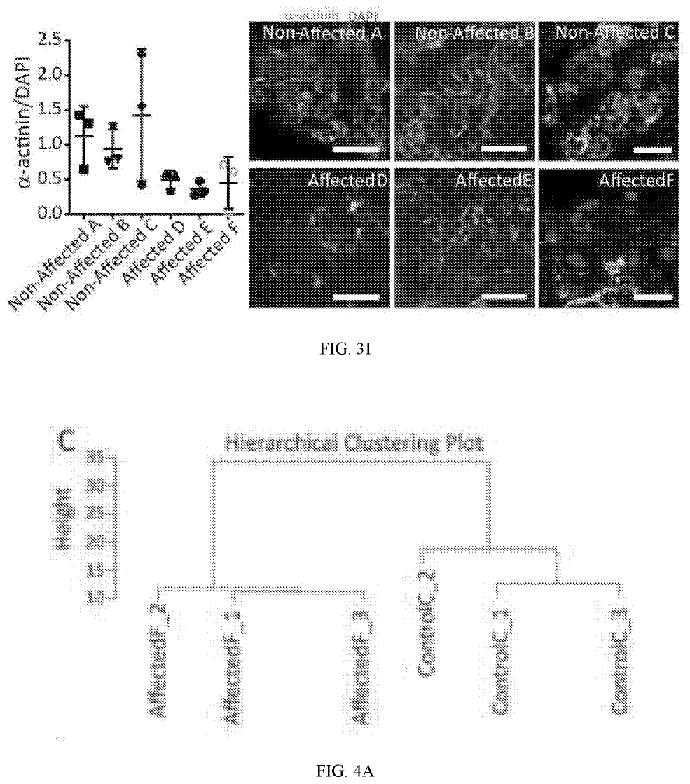

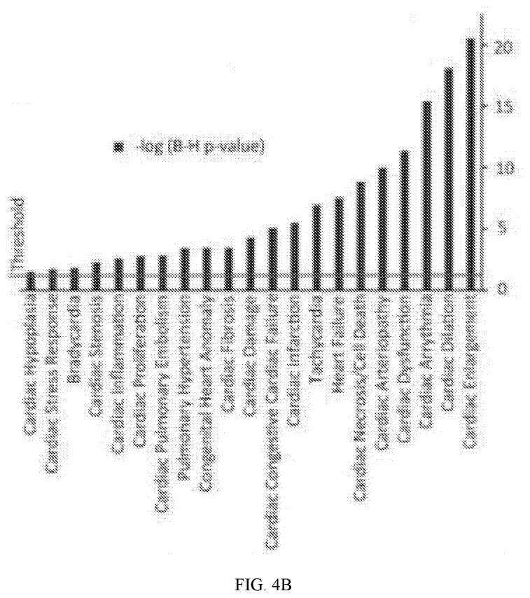

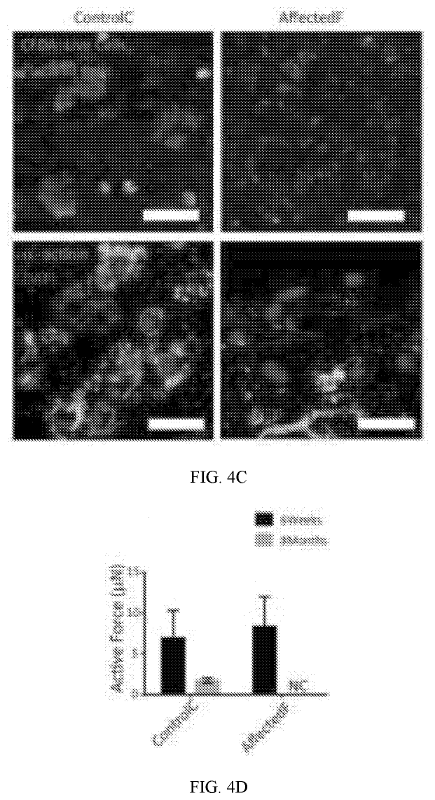

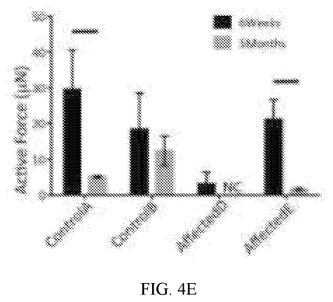

In certain embodiments the Biowire platform was used to generate a platform for disease modeling (FIGS. 1A, 1B, 3A-3I). As part of the disease model, iPSCs were obtained from patients enrolled in the NHLBI HyperGEN study, one of the largest epidemiological studies focusing on left ventricular hypertrophy (LVH) in families with primary hypertension. A comparison was made between in vitro ventricular tissues generated from iPSC-CMs obtained from hypertensive participants with clear echocardiographic evidence of left ventricular hypertrophy (referred to herein as the Affected group) versus ventricular tissues generated from iPSC-CMs obtained from participants without ventricular hypertrophy (referred to herein as the Non-Affected group), as summarized in FIG. 3A.

Although the underlying basis for the phenotypic differences between the Affected group and Non-Affected group is unknown, hypertension as well as the associated cardiac responses to the increased workloads generally represent a polygenic disorder. Thus, it was hypothesized that chronic electrical conditioning protocols, designed to mimic the chronic increases in cardiac workloads arising from hypertension, will uncover differences between the patient groups. Accordingly, tissues were conditioned during the first 6 weeks using previously-determined ventricular conditioning protocols. Thereafter, electrical stimulation was continued at 6 Hz for 1 additional week, after which the stimulation frequency was reduced to 3 Hz and maintained for up to 6 months (FIG. 3B) to mimic chronic increased workload, resulting in a total cultivation time of 8 months.

In contrast to previous modeling studies that focused on monogenic cardiac diseases, modeling of polygenic disease necessitates more comprehensive genetic profiling analysis. Interestingly, profiling of RNA expression in conditioned ventricular Biowires (FIG. 3C) after 8 months of culture demonstrates distinct gene expression profiles between the samples from the Affected versus Non-Affected patients as revealed from gene set enrichment analyses. These studies were performed in two separate batches of Biowires generated from cells from Affected and Non-Affected participants as summarized in FIG. 3C. In both batches, enrichment in 25 cardiac toxicity and canonical signaling pathways was consistently uncovered in Biowires from Affected vs Non-Affected patients (FIG. 3D), including many pathways broadly linked to pathological remodeling in cardiovascular disease such as cardiac enlargement, cardiac dilatation, cardiac dysfunction, heart failure, and cardiac hypertrophy signaling (FIG. 3C). Analysis of the specific genes related to cardiac hypertrophy and heart failure within individual replicates of independent experimental groups further indicates clear upregulation in all samples derived from the Affected participants (FIG. 3E), with only one of 3 replicates from one of the Non-Affected participants (Non-Affected A) exhibiting a relatively high expression of the hypertrophy-associated genes (FIG. 3E).

Consistent with these differences in mRNA expression, long-term culturing for 8 months led to profound differences in contractile function between Biowires from Affected participants compared to Non-Affected, with all 3 of the Affected samples generating virtually no force compared to the Non-Affected samples (FIGS. 3F, 3G). Despite these profound differences in contractile function, no differences in cell viability (FIG. 3H) or cardiomyocyte content (FIG. 3I) were observed in the Biowires from the two groups of participants.

Since cardiac hypertrophy in response to increased workload arising from conditions such as hypertension is a well-established risk factor for heart disease and failure (4), developing platforms to model these conditions would be highly desirable. Accordingly, Biowires were generated using ventricular cardiomyocytes created from iPSCs derived from participants enrolled in the NHLBI HyperGEN-LVH study (5), an established epidemiological cohort focusing on LVH and its underlying risk factors which started recruiting in 1996. Although all of these patients suffer from prolonged hypertension in association with marked elevations in cardiac workloads, they present with highly variable left ventricular hypertrophy. The blinded studies allowed the identification of three cell lines derived from affected hypertensive participants with the greatest amount of LVH, in comparison to those cell lines derived from unaffected participants with normal LV mass and contractile function. Specifically, Biowires generated from affected participants showed deterioration (or the complete absence) of active force generation after a prolonged culturing compared to the controls. This deterioration may be a result of cardiomyocyte death or disassembly of the contractile apparatus, both of which are consistent with progression towards heart failure. Remarkably, pathway analyses of RNA sequencing data from affected and control lines identified a significant enrichment in pathways linked to cardiac enlargement, cardiac dilatation, cardiac dysfunction, and heart failure. This model system may enable a fuller understanding of the disease mechanism responsible for progression from LVH to heart failure and may be used as a platform for future drug development.

In particular, in certain embodiments the hiPSCs and/or cardiomyocytes derived therefrom which are disclosed herein may be used for screening drug candidates. One or more hiPSCs and/or CMs from the Control/Non-Affected group may be combined with one or more hiPSCs and/or CMs from the Affected group may be combined as part of a kit and/or a method for screening drug candidates based on the procedures disclosed herein.

Each of the following references is incorporated by reference in its entirety for all purposes:
1. Hinson J T, Chopra A, Nafissi N, Polacheck W J, Benson C C, Swist S, et al. HEART DISEASE. Titin mutations in iPS cells define sarcomere insufficiency as a cause of dilated cardiomyopathy. Science. 2015 Aug. 28; 349(6251):982-6. PubMed PMID: 26315439. Pubmed Central PMCID: 4618316.
2. Wang G, McCain M L, Yang L, He A, Pasqualini F S, Agarwal A, et al. Modeling the mitochondrial cardiomyopathy of Barth syndrome with induced pluripotent stem cell and heart-on-chip technologies. Nature medicine. 2014 June; 20(6):616-23. PubMed PMID: 24813252. Pubmed Central PMCID: 4172922.
3. Aggarwal P, Turner A, Matter A, Kattman S J, Stoddard A, Lorier R, et al. RNA expression profiling of human iPSC-derived cardiomyocytes in a cardiac hypertrophy model. PloS one. 2014; 9(9):e108051. PubMed PMID: 25255322. Pubmed Central PMCID: 4177883.
4. Messerli F H, Rimoldi S F, Bangalore S. The Transition From Hypertension to Heart Failure: Contemporary Update. JACC Heart failure. 2017 August; 5(8):543-51. PubMed PMID: 28711447.
5. Williams R R, Rao D C, Ellison R C, Arnett D K, Heiss G, Oberman A, et al. NHLBI family blood pressure program: methodology and recruitment in the HyperGEN network. Hypertension genetic epidemiology network. Annals of epidemiology. 2000 August; 10(6):389-400. PubMed PMID: 10964005
6. Yu J, Vodyanik M A, Smuga-Otto K, Antosiewicz-Bourget J, Frane J L, Tian S, Nie J, Jonsdottir G A, Ruotti V, Stewart R, Slukvin I I, Thomson J A. Induced pluripotent stem cell lines derived from human somatic cells. Science. 2007 Dec. 21; 318(5858):1917-20. Epub 2007 Nov. 20.
7. Yu J, Hu K, Smuga-Otto K, Tian S, Stewart R, Slukvin I I, Thomson J A. Human induced pluripotent stem cells free of vector and transgene sequences. Science. 2009 May 8; 324(5928):797-801. doi: 10.1126/science. 1172482. Epub 2009 Mar. 26.
8. Kehat, I., Kenyagin-Karsenti, D., Snir, M., Segev, H., Amit, M., Gepstein, A., et al. (2001). Human embryonic stem cells can differentiate into myocytes with structural and functional properties of cardiomyocytes. Journal of Clinical Investigation, 108(3), 407-414. doi: 10.1172/JCI12131.
9. Xu, C., Police, S., Rao, N., & Carpenter, M. K. (2002). Characterization and enrichment of cardiomyocytes derived from human embryonic stem cells. Circulation Research, 91(6), 501-508.
10. He, J. Q., Ma, Y., Lee, Y., Thomson, J. A., & Kamp, T. J. (2003). Human embryonic stem cells develop into multiple types of cardiac myocytes: Action potential characterization. Circulation Research, 93(1), 32-39. doi: 10.1161/01.RES.0000080317.92718.9901.RES.0000080317.92718.99.
11. Mummery, C., Ward-van Oostwaard, D., Doevendans, P., Spijker, R., van den Brink, S., Hassink, R., et al. (2003). Differentiation of human embryonic stem cells to cardiomyocytes: Role of coculture with visceral endoderm-like cells. Circulation, 107(21), 2733-2740. doi: 10.1161/01.CIR.0000068356.38592.6801.CIR.0000068356.38592.68.
12. Sartiani, L., Bettiol, E., Stillitano, F., Mugelli, A., Cerbai, E., & Jaconi, M. E. (2007). Developmental changes in cardiomyocytes differentiated from human embryonic stem cells: A molecular and electrophysiological approach. Stem Cells, 25(5), 1136-1144. doi: 10.1634/stemcells.2006-0466.
13. Satin, J., Kehat, I., Caspi, O., Huber, I., Arbel, G., Itzhaki, I., et al. (2004). Mechanism of spontaneous excitability in human embryonic stem cell derived cardiomyocytes. Journal de Physiologie, 559(Pt 2), 479-496. doi: 10.1113/jphysiol.2004.068213jphysiol.2004.068213.
14. Zhang, J., Wilson, G., Soerens, A., Koonce, C., Yu, J., Palecek, S., et al. (2009). Functional cardiomyocytes derived from human induced pluripotent stem cells. Circulation Research, 104(4), e30-e41. doi: 10.1161/CIRCRESAHA.108.192237.
15. Zwi, L., Caspi, O., Arbel, G., Huber, I., Gepstein, A., Park, I. H., et al. (2009). Cardiomyocyte differentiation of human induced pluripotent stem cells. Circulation, 120(15), 1513-1523. doi: 10.1161/CIRCULATIONAHA.109.868885.

Thus, while the invention has been described above in connection with particular embodiments and examples, the invention is not necessarily so limited, and that numerous other embodiments, examples, uses, modifications and departures from the embodiments, examples and uses are intended to be encompassed by the claims attached hereto.

1. A method for generating an in vitro cardiac tissue model, comprising:
forming an elongated tissue by disposing a plurality of cardiomyocytes within a culture plate;
culturing the tissue such that each end of the elongated tissue contacts one of a pair of attachment wires adhered to the culture plate; and
electrically stimulating the elongated tissue in culture.

2. The method of claim 1, wherein electrically stimulating the elongated tissue in culture further comprises:
applying an electric field along a long axis of the elongated tissue.

3. The method of claim 2, wherein electrically stimulating the elongated tissue in culture further comprises:
applying the electric field at an initial frequency of 2 Hz;
incrementally increasing the electric field to a peak frequency of 6 Hz; and
decreasing the electric field to a maintenance frequency of 3 Hz.

4. The method of claim 3, wherein electrically stimulating the elongated tissue in culture further comprises:
applying the electric field at the initial frequency of 2 Hz for 1 week; and
incrementally increasing the electric field to a peak frequency of 6 Hz over a period of 4 weeks.

5. The method of claim 4, wherein electrically stimulating the elongated tissue in culture further comprises:
applying the electric field at the maintenance frequency of 3 Hz for six months.

6. The method of claim 1, further comprising measuring force generated by the elongated tissue by imaging movement of the attachment wires.

7. The method of claim 6, wherein the attachment wires comprise a POMaC polymer; and
wherein imaging movement of the attachment wires further comprises:
imaging movement of the attachment wires using UV light illumination and visible light detection.

8. The method of claim 1, wherein forming the elongated tissue further comprises:
disposing cardiac fibroblasts along with the cardiomyocytes within the culture plate.

9. The method of claim 1, wherein forming the elongated tissue further comprises:
disposing mesenchymal stem cells along with the cardiomyocytes within the culture plate.

10. The method of claim 1, wherein forming the elongated tissue further comprises:
disposing a hydrogel along with the cardiomyocytes within the culture plate.

11. The method of claim 1, wherein the cardiomyocytes comprise human induced pluripotent stem cells (hiPSCs).

12. The method of claim 11, wherein the hiPSCs are derived from a human subject with evidence of a cardiac disease.

13. The method of claim 12, wherein the cardiac disease is a polygenic disease.

14. The method of claim 13, further comprising:
analyzing gene expression in the elongated tissue to identify at least one gene related to the polygenic disease.

15. The method of claim 12, wherein the hiPSCs are at least one of: Affected D (no. A2637), Affected E (no. A2614), or Affected F (no. A2779).

16. The method of claim 11, wherein the hiPSCs are derived from a human subject without evidence of a cardiac disease.

17. The method of claim 16, wherein the hiPSCs are at least one of: Non-Affected A (no. A7156), Non-Affected B (no. 50000395), or Non-Affected C (no. U2474).

18. (canceled)

19. A kit for generating an in vitro cardiac tissue model, comprising:
a culture system including:
a culture plate and a pair of attachment wires comprising a POMaC polymer,
the culture plate comprising a pair of electrodes associated with the culture plate to apply an electric field along a long axis of a tissue within the culture plate;
a plurality of hiPSC-derived cardiomyocytes from at least one of a human subject with evidence of a cardiac disease and a human subject without evidence of a cardiac disease; and
a plurality of cardiac fibroblasts disposed in the culture plate with the plurality of hiPSC-derived cardiomyocytes.

20. The kit of claim 19, wherein the hiPSC-derived cardiomyocytes are from a human subject with evidence of a cardiac disease, the hiPSC-derived cardiomyocytes being selected from the group consisting of: Affected D (no. A2637), Affected E (no. A2614), and Affected F (no. A2779).

21. The kit of claim 19, wherein the hiPSC-derived cardiomyocytes are from a human subject without evidence of a cardiac disease, the hiPSC-derived cardiomyocytes being selected from the group consisting of: Non-Affected A (no. A7156), Non-Affected B (no. 50000395), and Non-Affected C (no. U2474).

22-54. (canceled)
